(19) United States
(12) Patent Application Publication
(10) Pub. No.: US 2005/0255133 A1
(43) Pub. Date: Nov. 17, 2005

(54) Topical composition for acne treatment

(75) Inventors: Joseph Schwarz, Markham (CA); Michael Weisspapir, Markham (CA)

Correspondence Address: MCCARTHY TETRAULT LLP, SUITE 4900, P.O. BOX 48, 66 WELLINGTON ST. WEST, TORONTO, ON, M5K 1E6, CA

(73) Assignee: ALPHARX INC., Markham (CA)

(21) Appl. No.: 10/842,524

(22) Filed: May 11, 2004

(51) Int. Cl. A61K 31/192; A61K 7/00

(52) U.S. Cl. 424/401; 514/568

(57) ABSTRACT

A topical composition for treatment of skin disorders such as acne is disclosed. The composition includes benzoyl peroxide in an amount from between 0.5 and 20% by weight. There is also provided a water miscible solvent for solubilizing the benzoyl peroxide in an amount from between 10% and 95% by weight and finally, a water miscible or water dispersible surfactant is present in an amount from between 0.5 and 95% by weight.

### FIELD OF THE INVENTION

The present invention relates to a topical formulation of benzoyl peroxide (BP) in solubilized form, suitable for the topical treatment of susceptible skin conditions such as acne, fungal infections, and the elimination of allergen(s) from the skin's surface. A method of manufacture of a stable topical solution of benzoyl peroxide is also set forth.

### BACKGROUND OF THE INVENTION

The invention encompasses a composition for solubilizing and stabilizing benzoyl peroxide for use in formulating dermatological, cosmetic, toiletry and personal care products, in order to increase the efficacy of these products. The composition includes the combination of anhydrous water-miscible solvents and a compatible surfactant to provide a stable solution of benzoyl peroxide. The use of benzoyl peroxide for treating acne, fungal infections, skin pigmentation, warts, or skin-related problems is well known in the preparation of cosmetic and dermatologic formulations. Benzoyl peroxide exists in crystalline form and is not satisfactorily soluble in water or oils traditionally used in cosmetic and dermatological preparations. A characteristic problem which occurs when using benzoyl peroxide in cosmetic and dermatologic preparations is that it tends to crystallize out of the various compositions, thus significantly reducing its bioavailability for the treatment or prevention of the aforementioned skin conditions. Furthermore, traditional benzoyl peroxide formulations form crystals on standing and precipitate out of the composition creating an unpleasant texture and appearance.

The cause of acne is not completely understood, but treatment of the condition includes a variety of different approaches, namely physical, mechanical, and pharmacological. According to the most popular hypothesis, acne is caused by a superficial microbial or fungal infection of the skin and is characterized by an excessive production of sebum from the sebaceous glands, the formation of pimples and comedones (blackheads) and the accompanied inflammation of the blemish spots. The usual results are papules, pustules or cysts often contaminated with bacteria, which cause secondary infections.

Accordingly, most anti-acne preparations can be divided in several groups, based on the mechanism of action:
a) Keratolytics; these function to reopen the duct once it has become blocked. Representative examples include hydroxyacids, sulphur, enzymes, azelaic acid
b) Anti-inflammatory agents; typical of which are salicylic acid, adapalene
c) Sebum production suppressors; examples include colloidal sulphur
d) Topical antibiotics; including erythromycin, clyndamycin
e) Systemic antibiotics; including tetracycline, minocyclin
f) Retinoids; such as Retin-A, retinoic acid; and
g) Topical antimicrobial drugs (antiseptics). These include chlorhexidine, hexetidine, benzalconium chloride, cetrimide, hydrogen peroxide and benzoyl peroxide.

Benzoyl peroxide is one of the most widely used agents for acne treatment, because of its successful combination of high antimicrobial activity, mild keratolytic (duct opening) properties and low cost. It acts in a combination of ways including a topical antimicrobial against the infecting bacteria, removal of the horny layer of skin, thickened sebum and the debris clogging the follicular openings.

There remains a need for solubilizing benzoyl peroxide for use in cosmetic and dermatological products to increase product efficacy.

Benzoyl peroxide is a white crystalline material that is stable at room temperature. It possesses strong oxidizing properties. Finely divided benzoyl peroxide is often incorporated into semisolid compositions for the convenience of application to the skin. However, due to the powerful oxidizing properties of benzoyl peroxide, the oxidation may result in unstable compositions demonstrating a rapid loss of activity in conventional ointments or cream bases.

A stable benzoyl peroxide composition that is effective in the treatment of acne and that has a projected shelf life of eight years or more, is described in U.S. Pat. No. 3,535,422, issued Oct. 20, 1970 to Cox et al. This patent describes a uniform distribution of finely dispersed benzoyl peroxide particles in an emulsion of water and certain emollients. When the composition is applied to the human skin, the water content of the emulsion evaporates leaving most of the emollients and the benzoyl peroxide crystals on the surface of the skin near and in contact with the acne sites.

Benzoyl peroxide has the potential to irritate the skin when applied at concentrations suitable for effective acne treatment. Combining it with commonly used surfactants, such as sodium lauryl sulphate, may exaggerate the skin irritation. To decrease irritability, moisturizers and emollients may be used in combination with mild non-ionic surfactants.

U.S. Pat. No. 4,056,611, issued Nov. 1, 1977 to Young, describes compositions containing a non-ionic surfactant and a short chain of alcohol and water. In U.S. Pat. No. 4,725,429, issued Feb. 16, 1988, Scott et al. present a composition of benzoyl peroxide in silicon oil combined with surfactants and high-alkyl alcohols, to provide increased bioavailability due to the occlusive properties of the vehicle.

The extreme low solubility of benzoyl peroxide in a water medium motivated investigators to develop solubilized formulations which provide a high concentration of the oxidizer at the point of action, i.e. clogged follicle or blemish spot. U.S. Pat. No. 4,923,900, issued May 8, 1990 to De Villez, discloses compositions containing benzoyl peroxide, water and a water miscible solvent having a boiling point greater than that of water. Dimethylisosorbide is indicated as a particularly useful solvent in the disclosed compositions. The disclosed compositions are indicated for the treatment of skin conditions such as acne and seborrhoea, dermatophyte infection, reactions to irritative plant contactants such as the oleoresins of poison ivy, and in the prevention of the development of offensive body odour. Nevertheless, in formulations prepared according to U.S. Pat. No. 4,923,900, only a small percentage of the benzoyl peroxide remains in a dissolved state, explaining why the treatment efficacy is much lower than would be indicated for totally dissolved oxidant.

Few examples of solubilized benzoyl peroxide formulations exist. Decker et al, in U.S. Pat. No. 4,925,666, issued May 15, 1990, describe a solubilized benzoyl peroxide formulation in polymeric siloxanes at an elevated temperature. Benzoyl peroxide forms a clear solution, but for cosmetic applications, it requires high concentrations of volatile organic solvents, such as acetone and isopropyl alcohol. Popp and Stiefel, in U.S. Pat. No. 6,433,024, issued Aug. 13, 2002, disclose a composition of benzoyl peroxide in an isosorbide ester and water vehicle containing surfactants, hydroxyacids and polyvinylpyrrolidone carboxylate to improve solubility and decrease irritation. None of the known current topical formulations of benzoyl peroxide possesses the optimal combination of effective anti-acne properties, a non-irritating composition and a formulation stability that provides for the satisfactory treatment of acne conditions.

An objective of the present invention is to provide a new and improved delivery system with higher efficacy of the active ingredient through increased transcutaneous penetration and bioavailability.

A further objective of the present invention is to provide an effective topical formulation for the treatment of acne and related skin conditions, comprised of solubilized benzoyl peroxide which does not precipitate out after application to the treatment site. Benzoyl peroxide is solubilized and stabilized within the cosmetic and dermatological formulations by the use of anhydrous solvents in combination with surfactants and some additives.

### DETAILED DESCRIPTION OF PREFERRED EMBODIMENTS

The present invention relates to a topical composition for skin treatment generally composed of a water-miscible solvent, benzoyl peroxide and a surfactant. Benzoyl peroxide in the composition persists in a dissolved state and does not precipitate out during storage or upon application to the skin. Additionally, the composition of the invention may optionally contain a viscosity regulation agent to provide a semi-solid consistency when required. A water absorption material can also be added to prevent precipitation of the benzoyl peroxide. This invention is intended for the treatment of a patient afflicted with acne or fungal skin infections by applying the compositions of this invention to the damaged areas of the patient's skin.

Benzoyl peroxide is a strong oxidizing agent which may be used in topical compositions for treating acne. However, in view of the low solubility in the presence of water when applied at concentrations that are effective for treating acne, benzoyl peroxide precipitates into a crystalline form. Precipitation causes a loss of activity and the presence of the formed crystals on the skin surface can be irritating. It is highly desirable to avoid precipitation and keep the active benzoyl peroxide in solution even after application to the skin, in order to provide a significant penetration into the skin and to develop an effective concentration in the targeted areas.

Some of the existing benzoyl peroxide gels, containing high levels of alcohol, propylene glycol or a mixture thereof, form clear solutions. However, upon contact with the skin, large amounts of water are absorbed from the skin due to the hypertonicity of the gel base and most of the dissolved benzoyl peroxide precipitates onto the skin surface, loosing its activity.

It has been found that the use of a water-miscible solvent with an appropriate surfactant (in various ratios), prevents benzoyl peroxide precipitation and provides high solution stability during a variety of storage conditions.

Any pharmaceutically acceptable surfactants may be included in the topical compositions of the present invention. Such surfactants include polyethoxylated aliphatic or aromatic derivatives, which are stable in the presence of such strong oxidative materials such as benzoyl peroxide, e.g., polysorbates (Tween®-20, -40, -60, ICI), ethoxylated derivatives of hydrogenated castor oil (Cremophor® RH-40, BASF), ethoxylated derivatives of hydrogenated sterols, such as lanolin (Lipolan® 31-20, Lipo Chemicals); ethoxylated cholesterol (Choleth-24, RITA); polyglycerin derivatives (Cremophor® GS 32, BASF; Plurol Stearique WL 1009, Gattefosse), sugar esters (sucrose stearate (Crodesta®, Croda) and polyol esters or ethers with HLB in the range of between 3 and 24.

Preferably, the surfactant is a polyethoxylated sterol derivative, e.g. lanolin or cholesterol based. The amount of surfactant present in the compositions of the invention may vary from 5% to about 95% by weight, of the composition. Preferably, the surfactant is present in an amount from about 10% to about 40% by weight.

The compositions of the present invention may also contain other ingredients that are commonly included in topical pharmaceutical compositions. Such ingredients include thickeners, preservatives, binders, water absorbents and opacifiers.

Isosorbides, which may be included in the topical compositions of the present invention, include any pharmaceutically acceptable isosorbide—e.g. dimethyl isosorbide, diethyl isosorbide, and ethylmethyl isosorbide. Preferably, the isosorbide is an alkyl ester of isosorbide, such as dimethyl isosorbide. The amount of isosorbide present in the compositions of the invention may vary from about 1% to about 95% by weight, of the composition. Preferably, the isosorbide is present in an amount from about 5% to about 50% by weight.

Thickeners which may be used in the topical compositions of the present invention include any pharmaceutically acceptable thickener—e.g. cetostearyl alcohol, microcrystalline cellulose, powdered cellulose and cellulose derivatives, starch and starch derivatives, polyethylene glycol, xanthan gum, calcium silicate (e.g., Huberderm®, J. M. Huber Corp., MD, USA) and magnesium aluminium silicate. The thickeners may be present in the compositions of the invention in an amount from about 0.5% to about 60% by weight, of the composition. Preferably, the thickener is present in total amount from about 1% to about 30% by weight.

Benzoyl peroxide itself possesses strong antibacterial properties and usually does not require the addition of preservatives. If necessary, any pharmaceutically acceptable preservative, stable to oxidation (e.g., Bronopol™, Benzalconium chloride, Quaternium-15), may be included in the topical compositions of the present invention. The amount of preservatives present in the compositions of the invention may be from about 0.05% to about 2% by weight, of the composition.

Having thus described the invention, reference will now be made to the accompanying examples.

### EXAMPLE 1

1.4 g of wetted benzoyl peroxide (75% B₂O₂, 25% water) was dissolved in mixture of 2.4 g (11.7% of final weight) DMIS (Dimethyl isosorbide (Arlasolve® DMI, Uniqema, DE, USA) and 5 g (24.4%) dipropylene glycol dibenzoate, Finsolv® PG-22, Fintex Inc., NC, USA) at 45° C. After dissolving 5 g (24.4%) of cetostearyl alcohol (Croda Inc., NJ, USA), 0.2 g (1%) of Brij®-78P (eicosaethylene glycol octadecyl ether, Fluka, Switzerland), 0.1 g (0.5%) of Forlan® C-24 (Choleth-24 and Ceteth-24, RITA, IL, USA) are added. The mixture was heated to 45° C. and 6.4 g (31.2%) of hot water (75-80° C.) was added with intensive mixing. The resultant cream (5.1% of anhydrous benzoyl peroxide by weight) was cooled, packaged in a suitable air-tight container and stored at room temperature.

### EXAMPLE 2

Example 2 was prepared in a similar manner to that of example 1, but was comprised of 6.3% DMIS, 6.3% Glycofurol-75 (Tetragydrofurfuryl alcohol polyethylene glycol ether, Sigma-Aldrich, MO, USA), 12.5% Finsolv® PG-22, 12.5% Finsolv® TN-O (C12-15 Alkyl Benzoate, Fintex, NC, USA), 12.5% Cetostearyl alcohol, 1.5% Cholesterol (Sigma-Aldrich, MO, USA), 1% Forlan® C-24, 0.3% Tween®-20 (Polysorbate-20, Sigma-Aldrich, MO, USA), Water 38.8%. Subsequent to the cream preparation, 1.5% of colloidal silicon dioxide (Cab-O-Sil® M5, Cabot, USA) was added.

### EXAMPLE 3

Example 3 was prepared following the procedures of example 2, but Finsolv® TN-O was totally replaced with Finsolv® PG-22 (25% by weight).

### EXAMPLE 4

Example 4 followed the preparation steps of example 1, but was comprised of 6.3% DMIS, 6.3% Glycofurol-75 (Tetragydrofurfuryl alcohol polyethylene glycol ether, Sigma-Aldrich, MO, USA), 25% Finsolv® PG-22, 9% Cetostearyl alcohol, 10% Precirol® ATO (Glycerol Distearate, Gattefosse, France), 1.5% Forlan® C-24, Water 35%.

### EXAMPLE 5

Example 5 preparation followed example 4, but additionally contained 3.8% of cross-linked polyvinylpyrrolidone (Polyplasdone® XL-10, ISP Technologies, NJ, USA) and 0.3% of Cyclopentasiloxane (Si-Tec™ CM-040, ISP Technologies, NJ, USA).

### EXAMPLE 6

Example 6 is similar in composition to example 4, but contained no DMIS. Precirol® ATO was replaced with Myvaplex™ 600 (Glyceryl stearate NF, Eastman, USA), 22.5% by weight.

### EXAMPLE 7

Example 7 is similar in composition to example 4, but contained no Glycofurol-75, Finsolv® PG-22 was entirely replaced with Finsolv® TN-O (23.6% by weight), and Precirol® ATO was replaced with Myvaplex™ 600 (Glycerine monostearate, Eastman Chemicals, TN, USA), 21.2% by weight.

### EXAMPLE 8

1.4 g of wet Benzoyl peroxide (75% BzO2, 25% water) was dissolved in a mixture of 7.0 g (35% of final weight) DMIS and 3 g (15%) of Finsolv® PG-22, Fintex Inc., NC, USA). After dissolving 1.8 g (9%) of cetostearyl alcohol (Croda Inc., NJ, USA), 1.5 g (7.5%) of Finsolv® 137 (Benzoyl behenate, Fintex Inc., NC, USA), 0.6 g (3%) of Forlan® C-24 is added. The mixture was heated to 45° C. and 4.7 g (23.5%) of hot water (75-80° C.) was added with intensive mixing. The cream obtained (5% of anhydrous benzoyl Peroxide by weight) was cooled, packaged into a suitable air-tight container and stored at room temperature.

### EXAMPLE 9

1.4 g of wet Benzoyl peroxide (75% B₂O₂, 25% water) was dissolved in a mixture of 3.5 g (17.5% of final weight) DMIS and 3.8 g (19%) of Finsolv® PG-22, Fintex Inc., NC, USA) at 50° C. After dissolving 1.5 g (9%) of Cetostearyl Alcohol, 1.5 g (7.5%) of Finsolv® 137 (Benzoyl behenate, Fintex Inc., NC, USA), 1.7 g (8.5%) of Precirol® ATO, 0.3 g (1.5%) of Forlan® C-24, 50 mg (0.25%) of Cyclopentasiloxane (Si-Tec™ CM-040) was added. The mixture was heated to 45° C. and 7 g (35%) of hot water (75-80° C.) was added with intensive mixing. After cooling, 0.4 g (3.5%) of cross-linked polyvinylpyrrolidone (Polyplasdone® XL-10) was added and mixed thoroughly. The cream obtained (5% of anhydrous benzoyl Peroxide by weight) was cooled and packaged into a suitable air-tight container and stored at room temperature.

### EXAMPLE 10

1.5 g of wet Benzoyl peroxide (75% BzO2, 25% water) was dissolved in a mixture of 5 g (22.1% of final weight) DMIS and 3 g (13.3%) of Ethoxydiglycol (Diethylene glycol monoethyl ether, Transcutol® P, Gattefosse) 0.2 g (0.9%) of Finsolv® PG-22, Fintex Inc., NC, USA) at 45° C. After dissolving 2 g (8.9%) of Polyethylene glycol 4000 (Fluka), 6.5 g (28.8%) of Lipolan®-31 (PEG-24 Hydrogenated lanolin, LIPO Chemicals, NJ, USA) was added and the mixture was heated to 45° C. with occasional stirring until a clear solution formed. After cooling to 40° C., 0.4 g (1.8%) of calcium silicate (Huberderm™ 1000, J. M. Huber Corp., MD, USA) and 4.0 g of Aluminium Starch Octenylsuccinate (Dry-Flo® PC, National Starch and Chemical, NJ, USA) were added and mixed thoroughly. The ointment obtained (5% of anhydrous Benzoyl Peroxide by weight) was cooled and packaged into a suitable air-tight container and stored at room temperature.

### EXAMPLE 11

0.7 g of wet benzoyl peroxide (75% BzO2, 25% water) was dissolved in a mixture of 4.4 g (41.5% of final weight) of Ethoxydiglycol (Diethylene glycol monoethyl ether, Transcutol® P, Gattefosse) and 3 g (28.3%) of Finsolv® EMG-20 (Methylgluceth-20 Benzoate, Fintex Inc., NC, USA) at 40° C. After dissolving, the mixture was heated to 45° C., and 1 g (9.4%) of Benzoyl Behenate (Finsolv®137, Fintex), 0.5 g (4.7%) of Sucrose Stearate (SP-40C, Sisterna. C.V., The Netherlands) and 1 g (9.4%) of Fancol® LH-20 (PEG-20 Hydrogenated lanolin, The Fanning Corporation, Chicago, Ill., USA) were added with occasional stirring until a clear solution formed. The melted composition was cooled to room temperature while mixed constantly. The ointment obtained (5% of anhydrous benzoyl peroxide by weight) was packaged into a suitable air-tight container and stored at room temperature.

### EXAMPLE 12

Example 12 is similar in composition and preparation to example 11, but DMIS was entirely replaced with an equal amount of N-methylpyrrolidone (NMP, BASF)

### EXAMPLE 13

Example 13 is similar in composition and preparation to example 11, but Ethoxydiglycol (Transcutol®) was entirely replaced with an equal amount of Propylene Glycol NF (Merk)

### EXAMPLE 14

Example 14 is similar in composition and preparation to example 9, but cross-linked polyvinylpyrrolidone (Polyplasdone® XL-10) was entirely replaced with a mixture of 0.2 g (1.8%) of microcrystalline cellulose (Avicel-101, FMC, Ireland) and 0.2 g (1.8%) of Aluminium Magnesium Silicate (Neusilin®, Fuji, Japan)

### EXAMPLE 15

0.75 g of wet benzoyl peroxide (75% BzO2, 25% water) was dissolved in 5 g (25% of final weight) of ethyl alcohol (USP grade) at 60° C. After dissolving, 5 g (25%) of Polyethylene glycol 8000 (Fluka), 4 g (20%) of Cremophor® RH-40 (PEG-40 Hydrogenated castor oil, BASF) were added and the mixture was heated to 45° C. with occasional stirring, until a clear solution formed. After cooling to 40° C., 0.4 g (2%) of calcium silicate (Huberderm® 1000, J.M. Huber Corp., MD, USA) and 3.6 g (18%) of Aluminium Starch Octenylsuccinate (Dry-Flo® PC, National Starch and Chemical, NJ, USA) was added and mixed thoroughly. The ointment obtained (2.6% of anhydrous benzoyl peroxide by weight) was cooled, packaged into a suitable air-tight container and stored at room temperature.

### EXAMPLE 16

0.8 g of wet benzoyl peroxide (75% BzO2, 25% water) was dissolved in 2.5 g (33.3% of final weight) DMIS and 1 g (13.3%) of Ethoxydiglycol (Diethylene glycol monoethyl ether, Transcutol® P, Gattefosse). After dissolving, 0.4 g (5.3%) of Polyethylene glycol 8000 (Fluka), 1.6 g (21.3%) of Supersat® AWS-24 (Ethoxylated hydrogenated lanolin, RITA) were added and the mixture was heated to 45° C. with occasional stirring, until a clear solution formed. After cooling to 40° C., 0.1 g (1.3%) of calcium silicate (Huberderm® 1000, J.M. Huber Corp., MD, USA) and 1.1 g (14.7%) of Aluminium Starch Octenylsuccinate (Dry-Flo® PC, National Starch and Chemical, NJ, USA) was added and mixed thoroughly. The ointment obtained (8.0% of anhydrous benzoyl peroxide by weight) was cooled and packaged into a suitable air-tight container and stored at room temperature.

Penetration of Topical Formulations with Benzoyl Peroxide:

Comparative Investigaton

In order to compare the penetration of active benzoyl peroxide, 0.5 g of topical formulation was applied to a flat surface of 3% agar gel containing 1.0% potassium iodide and 0.1% corn starch. The interaction of potassium iodide with benzoyl peroxide causes the formation of free iodine. An intense dark blue color then develops due to the reaction of the free iodine with the corn starch. The distance from the gel surface to the color reaction front is used as an indicator of benzoyl peroxide penetration into the gel. Table 1 tabulates the data obtained.

TABLE 1
Penetration of benzoyl peroxide from topical
formulations into 3% agar gel (depth of color reaction front, mm)

| | | | | | | Solugel ® | | |
|---|---|---|---|---|---|---|---|---|
| | | | | | | STIEFEL | PersaGel ® | |
| Time, | Example | Example | Example | Example | Example | (U.S. Pat. No. | (Johnson & | U.S. Pat. No. |
| hours | 1 | 4 | 8 | 9 | 10 | 4,923,900) | Johnson) | 6,433,024 |
| 0 | 0 | 0 | 0 | 0 | 0 | 0 | 0 | 0 |
| 0.25 | 0.2 | 0.8 | 1.6 | 0.2 | 2.5 | 1 | 0 | 0.2 |
| 0.5 | 0.4 | 3 | 3.0 | 0.3 | 4.8 | 2.5 | 0 | 0.5 |
| 1 | 0.5 | 3.5 | 4.5 | 0.4 | 7 | 4 | 0 | 0.9 |
| 2 | 0.7 | 5 | 7 | 0.6 | 13 | 4.5 | 0 | 1.4 |
| 24 | 2.4 | 9 | 21.5 | 2 | 36 | 7.2 | 0.3 | 3.6 |

Microscopic investigations show the absence of benzoyl peroxide precipitate crystals at the interface after contact with the water phase for at least 10-20 minutes after contact, for majority of prepared formulations. This absence of precipitate formation explains the deep penetration of the peroxide into the gel described in the above investigation.

Although embodiments of the invention have been described above, it is limited thereto and it will be apparent to those skilled in the art that numerous modifications form part of the present invention insofar as they do not depart from the spirit, nature and scope of the claimed and described invention.

1. A topical composition for treatment of skin disorders, comprising:
benzoyl peroxide in an amount from between 0.5 and 20% by weight;
a water miscible solvent for solubilizing said benzoyl peroxide in an amount from between 10% and 95% by weight; and
a water miscible or water dispersible surfactant in an amount from between 0.5% and 95% by weight.

2. The composition of claim 1, wherein said water miscible solvent is selected from the group consisting of aliphatic alcohols, propylene glycol, butylene glycol, polyethylene glycols, ethoxydiglycol, isosorbide ethers—dimethylisosorbide, diethylisosorbide, methylethylisosorbide, propylene carbonate, acetamide, solketal, dimethylformamide, ethyllactate and N-methylpyrrolidone.

3. The composition of claim 1, wherein said water miscible or water dispersible surfactant is selected from the group consisting of dermatologically acceptable polyethoxylated aliphatic or aromatic derivatives, polyglycerin derivatives, sugar and polyol esters or ethers with HLB in the range of between 3 and 24.

4. The composition of claim 1, wherein said benzoyl peroxide is solubilized in said solvent.

5. The composition of claim 1, further including a thickener in amount from between 0.5% and 50% by weight.

6. The composition of claim 5, wherein said thickener prevents precipitation of benzoyl peroxide during storage.

7. The composition of claim 5, wherein said thickener is a dermatologically acceptable material with melting point of not less than 25° C.

8. The composition of claim 1, further including a moisture absorbing additive in amount from between 0.5 and 50% by weight.

9. The composition of claim 8, wherein said moisture absorbing additive is insoluble in water and in components of said topical composition.

10. The composition of claim 8, wherein said moisture absorbing additive selected from the group consisting of inorganic silicates, phosphates, carbonates, alumosilicates, ceolites; cross-linked polymers, polyvinylpyrrolidone PVP-XL, polysaccharides, powdered cellulose, cellulose fibres or microcrystalline cellulose, starch and starch derivatives, polyacrylates and fibrous materials.

11. The composition of claim 1, wherein said water miscible solvent is dimethylisosorbide, ethoxydiglycol, or mixture thereof in ratio from between 1:10 and 10:1.

12. The composition of claim 1, wherein said skin disorder is a viral infection.

13. The composition of claim 1, wherein said skin disorder is a microbial infection.

14. The composition of claim 1, wherein said skin disorder is a fungal infection.

15. A treatment method for a person afflicted with acne or superficial fungal infection, comprising applying to an affected area of the person's skin, a therapeutically effective amount of a composition comprising:
benzoyl peroxide in an amount from between 0.5 and 20% by weight;
a water miscible solvent for solubilizing said benzoyl peroxide in an amount from between 10% and 95% by weight;
a water miscible or water dispersible surfactant in an amount from between 0.5% and 95% by weight;
dermatologically acceptable thickener with a melting point no less than 25° C.; and
a dermatologically acceptable moisture absorbing additive in amount from between 0.5% and 50% by weight.

16. The method of claim 12, wherein said water miscible solvent is dimethylisosorbide, ethoxydiglycol, or a mixture thereof in a ratio from between 1:10 and 10:1.

17. A method for improving skin penetration of benzoyl peroxide in topical composition comprising:
providing benzoyl peroxide in an amount between 0.5 and 20% by weight;
a water miscible solvent for solubilizing said benzoyl peroxide in an amount from between 10% and 95% by weight;
a water miscible or water dispersible surfactant in an amount from between 0.5% and 95% by weight; and
mixing to form a liquid or semi-solid composition where said benzoyl peroxide is solubilized in said solvent.
